(19) United States
(12) Patent Application Publication
(10) Pub. No.: US 2025/0064313 A1
(43) Pub. Date: Feb. 27, 2025

(54) ALL-ROUND OBSERVATION UNDER-ACTUATED CAPSULE ROBOT AND AXIS ROLLING-OVER LOCOMOTION METHOD THEREOF BY MAGNETIC FIELD CONTROL

(71) Applicant: DALIAN UNIVERSITY OF TECHNOLOGY, Dalian , Liaoning (CN)

(72) Inventors: Yongshun ZHANG, Dalian, Liaoning (CN); Yan CUI, Dalian, Liaoning (CN); Hai DONG, Dalian, Liaoning (CN); Zhenhu LIU, Dalian, Liaoning (CN); Qing SHAN, Dalian, Liaoning (CN)

(21) Appl. No.: 18/014,629

(22) PCT Filed: Sep. 26, 2022
(86) PCT No.: PCT/CN2022/121215

§ 371 Date: Jan. 5, 2023

(30) Foreign Application Priority Data
Sep. 6, 2022 (CN) 2202211084837.X

(51) Int. Cl. A61B 1/273; A61B 1/00; A61B 1/04

(52) U.S. Cl. CPC A61B 1/273; A61B 1/00158; A61B 1/041

(57) ABSTRACT

The present invention discloses an all-round observation under-actuated capsule robot and an axis rolling-over locomotion method thereof by magnetic field control. A radially magnetized NdFeB magnet ring is loaded into an inner cavity of a capsule sphere in a non-connected way, and the NdFeB magnet ring independently idles with the rotating magnetic field around a capsule axis and is completely suspended in the inner cavity filled with silicone oil in the capsule sphere. Under the drive of a coaxial following magnetic moment, the under-actuated capsule sphere cannot rotate around the capsule axis, but the capsule axis can roll synchronously with the rotation axis of the magnetic field. The present invention increases the scanning range of the capsule, enhances environmental adaptability, and has high accuracy of fixed point posture adjustment of the capsule, rolling locomotion stability, good visual observation effect and good application prospect.

### TECHNICAL FIELD

The present invention belongs to the technical field of automation engineering, relates to an all-round observation under-actuated capsule robot and an axis rolling-over locomotion method thereof by magnetic field control, and in particular to a locomotion method by magnetic field control for all-round observation and diagnosis and treatment in scenarios of the gastrointestinal (GI) tract by a way of planning an axis rolling-over motion track of an overall under-actuated capsule robot through a coaxial following magnetic moment effect generated by a space universal rotating magnetic field (SURMF).

### BACKGROUND

With the acceleration of the pace of social development, gastrointestinal diseases have been increased year by year, and have seriously threatened human health. Early diagnosis of the gastrointestinal diseases by endoscopes can effectively improve the cure rate, but the traditional interposition gastrointestinal endoscopes are complicated in operation, arouse discomfort, psychological fear and even harm to patients, thus are not suitable for large-scale screening of the gastrointestinal diseases.

Painless capsule endoscope is a boon to patients, and becomes the only means of non-invasive examination in the small intestine. However, the passive capsule endoscope currently used clinically mainly relies on gravity and gastrointestinal peristalsis to move. The posture of the capsule endoscope in 3D regions of the gastrointestinal tract is random, and cannot be controlled by a doctor to observe key areas of the gastrointestinal tract, resulting in the problems of high missed rate and low inspection efficiency. Thus, the passive capsule endoscope is still limited clinically.

In view of the problems existing in the passive capsule endoscope, it is urgent to develop an active capsule robot in order to realize the active control of its e locomotion and posture, which is expected to improve the inspection efficiency in 3D regions of the stomach and colon and lay a foundation for targeted drag delivery and sampling.

At present, a wireless active capsule robot with embedded magnet driver mainly uses two driving modes of an external permanent magnet and an electromagnetic device to achieve capsule motion and posture control. Although the driving mode of the external permanent magnet solves the problem of wireless power supply of the capsule robot and achieves clinical application, the precise control conditions of capsule motion and posture through the external permanent magnet are harsh and difficult to implement. The reason is that the external permanent magnet generates a gradient magnetic field, and the capsule is simultaneously subjected to magnetic force and magnetic moment. The magnetic force and the magnetic moment generated by the gradient magnetic field are coupled with each other, and are decreased with distance r from the capsule to the external permanent magnet in an exponential rule of 1/r⁴ and 1/r³, respectively. Because it is difficult to detect and control in real time the distance r between the capsule inside human body and the magnet during clinical operation, the control of static balance of the capsule is difficult due to the wide range variation of the magnetic force and the magnetic moment.

Firstly, the safety of realizing capsule position control through static magnetic force balance of the permanent magnet is poor. Because the magnetic force exerted on the capsule changes exponentially with the distance r from the capsule to a magnetic source, a small change of the distance from the magnetic source to the capsule robot will seriously affect the change of the magnetic force. For patients with large change in body fat rate, the capsule robot will lose balance due to the sudden change of the magnetic force, and has a risk of impacting the inner wall of the organ. In addition, the coupling of the magnetic force and the magnetic moment also increases the difficulty of magnetic levitation balance control.

Secondly, capsule posture control realized by the static magnetic moment balance of a permanent magnet is of high randomness. As the distance between the capsule and the external permanent magnet is increased, the magnetic force on the capsule decays one order of magnitude faster than the magnetic moment. Therefore, when the capsule reaches a large and appropriate distance r from the external permanent magnet, the influence of the magnetic force can be ignored. It can be approximated that the capsule is only under the action of the magnetic moment, and the decoupling of the magnetic force and the magnetic moment under the gradient magnetic field is realized. Obviously, capsule posture control under static balance of pure magnetic moment is easier. However, when the distance r is large, the magnetic moment decays greatly, and even the magnetic moment is not enough to overcome the viscous damping of the GI tract, resulting in failure of posture control. Clinically, in order to achieve the internal detection of the stomach, operators must overcome gravity by using the buoyancy generated by the liquid drunk by the patient, so that the capsule is in a state of force balance in the liquid. Then, not only the position control of the capsule suspended in the liquid is realized through micro magnetic force, but also free and flexible adjustment of the posture of the capsule suspended in the liquid is implemented through small magnetic moment. The suspended capsule is not in contact with the gastric wall, which avoids the failure of the capsule posture control caused by that the magnetic moment is insufficient to overcome the viscous damping of the GI tract. However, because the capsule uses magnetic levitation control under static balance, although the liquid in the stomach greatly hinders the stimulus movement due to sudden change of the magnetic force of the capsule to effectively avoid the risk caused by the impact on the inner wall of the organ, to find appropriate position and azimuth angle to observe specific regions in the stomach, specialized operators must rely on their experience and repeatedly adjust the position and azimuth of the external permanent magnet to establish posture control under the static magnetic moment balance of the capsule, which is difficult for clinical popularization and promotion. It can be seen that the control of the capsule posture by the external permanent magnet is inefficient and difficult. Especially when the capsule is tightly enveloped by gastrointestinal tissue, the magnetic moment is not large enough to overcome the resistance of the tightly enveloped capsule by the gastrointestinal tissue, resulting in the failure of posture control.

Finally, the capsule posture sometimes has non-uniqueness. The magnet embedded in the capsule robot has a singular plane inside which the capsule posture has non-uniqueness and the capsule posture is out of control. Especially in 3D regions of the gastrointestinal (GI) tract, it is more difficult to control the posture and motion of the capsule by the external magnet. The above factors restrict the clinical application and promotion of external permanent magnet driven capsules.

In conclusion, although the inconvenience is caused by drinking the liquid by the patient, according to the decoupling characteristics and long-distance attenuation features of the magnetic force and the magnetic moment of the external permanent magnet, only magnetic moment drive can effectively control the position and the posture of the capsule by means of liquid buoyancy, and can ensure the safety. Although the efficiency of posture adjustment is low, the driving mode of the magnetic moment provides a new way to explore the effective posture control of the capsule robot.

Compared with the gradient magnetic field generated by the external permanent magnet, the magnitude and the orientation of the electromagnetic field can be digitally controlled by the current. The adjustment of the magnetic field is more flexible and convenient. In particular, the space universal uniform rotating magnetic field can be generated by the superposition of the ti-axis orthogonal square Helmholtz coils (TOSHC). The magnet embedded in the capsule only produces pure coupled magnetic moment in the uniform rotating magnetic field. The magnetic moment within the uniform region of the coils is constant and has no attenuation; the decoupling of the magnetic force and the magnetic moment is fully realized, that is, the influence of the magnetic force on the magnetic moment is eliminated; the pure magnetic moment is used for controlling the posture of the capsule, which is high in accuracy, good in flexibility and good in safety; and it is expected to solve the problems of posture and motion control of the capsule in the 3D regions of the gastrointestinal tract.

To overcome the limitations of static balance control of the capsule posture by the permanent magnet and in view of the advantages of pure magnetic moment control, a dynamic balance control scheme of capsule posture control by the pure coupled magnetic moment of the SURMF is proposed. At the same time, a coaxial following magnetic moment effect is discovered. That is, when a radially magnetized cylindrical magnet rotates with the SURMF, its axis direction always follows and eventually remains consistent with the direction of the normal vector of the rotating magnetic field (the rotation axis of the magnetic field). The coaxial following magnetic moment effect not only provides a new way to explore the drive of a space universal motor, but also lays a foundation for the accurate control of the capsule posture. In the invention patent “an electromagnetic drive spherical robotic wrist with two degrees of freedom and a control method thereof” (patent authorization ZL202010484343.5) applied by Dalian University of Technology, the mathematical proof of “coaxial following magnetic moment effect” is given in detail as follows:

o-x₀y₀z₀ is a ground fixed coordinate system. It is assumed that a radial permanent magnet placed in a rotating magnetic field at the initial time remains horizontal, and its central axis n_{B} coincides with y₀ axis, as shown by position 1 in FIG. 1; the magnetic moment vector of a radially magnetized permanent magnet is m₁, and the radially magnetized permanent magnet rotates synchronously around the central axis under the action of the rotating magnetic field; angular velocity is ω, and m_{B} is the modulus of the magnetic dipole moment of the radially magnetized permanent magnet. Then, the magnetic moment vector m₁ of the radially magnetized permanent magnet in the rotation process can be expressed in the coordinate system o-x₀y₀z₀ as:

$${m}_{1} = [ \begin{matrix}cos ( ω t ) & 0 & sin ( ω t ) \\ 0 & 1 & 0 \\ - sin ( ω t ) & 0 & cos ( ω t )\end{matrix} ] [ \begin{matrix}0 \\ 0 \\ {m}_{B}\end{matrix} ] = [ \begin{matrix}{m}_{B} sin ( ω t ) \\ 0 \\ {m}_{B} cos ( ω t )\end{matrix} ] \qquad ( 1 )$$

At this moment, the SURMF in the direction of n_{f}(swing angle θ=0, and pitch angle δ=λ) is applied. The amplitude of a rotating magnetic vector is B₀, and the rotating magnetic vector B₁ can be expressed in the coordinate system o-x₀y₀z₀ as:

$${B}_{1} = [ \begin{matrix}1 & 0 & 0 \\ 0 & cos λ & - sin λ \\ 0 & sin λ & cos λ\end{matrix} ] [ \begin{matrix}{B}_{0} sin ( ω t ) \\ 0 \\ {B}_{0} cos ( ω t )\end{matrix} ] = [ \begin{matrix}{B}_{0} sin ( ω t ) \\ - {B}_{0} cos ( ω t ) sin λ \\ {B}_{0} cos ( ω t ) cos λ\end{matrix} ] \qquad ( 2 )$$

According to a vector cross product formula, the magnetic moment received by the radially magnetized permanent magnet in the SURMF is:

$$T = [ \begin{matrix}{T}_{x} \\ {T}_{y} \\ {T}_{z}\end{matrix} ] = {m}_{1} \times {B}_{1} = [ \begin{matrix}{m}_{B} sin ( ω t ) \\ 0 \\ {m}_{B} cos ( ω t )\end{matrix} ] \times [ \begin{matrix}{B}_{0} sin ( ω t ) \\ - {B}_{0} cos ( ω t ) sin λ \\ {B}_{0} cos ( ω t ) cos λ\end{matrix} ] = [ \text{} \begin{matrix}{m}_{B} {B}_{0} {cos}^{2} ( ω t ) sin λ \\ - {m}_{B} {B}_{0} sin ( 2 ω t ) ( cos λ - 1 ) / 2 \\ - {m}_{B} {B}_{0} cos ( ω t ) sin ( ω t ) sin λ\end{matrix} ] = [ \begin{matrix}{m}_{B} {B}_{0} ( 1 + cos ( 2 ω t ) ) sin λ / 2 \\ - {m}_{B} {B}_{0} sin ( 2 ω t ) ( cos λ - 1 ) / 2 \\ - {m}_{B} {B}_{0} sin ( 2 ω t ) sin λ / 2\end{matrix} ] \qquad ( 3 )$$

From formula (3), the magnetic moments Tₓ, T_{y}, T_{z} in all directions received by the radially magnetized permanent magnet in the SURMF are all periodic functions, and the periods are respectively: tₓ=t_{y}=t_{z}=π/ω; the pitch angle λ at the initial position is a constant value; at this moment, the average moments received by the radially magnetized permanent magnet in all directions within a single period are:

$$\{ \begin{matrix}{\overline{T}}_{x} = \frac{1}{2} {m}_{B} {B}_{0} sin λ + \frac{1}{2} {m}_{B} {B}_{0} sin λ {\int }_{- \frac{π}{2 ω}}^{\frac{π}{2 ω}} cos ( 2 ω t ) dt = \frac{1}{2} {m}_{B} B sin λ \\ {\overline{T}}_{y} = - \frac{1}{2} {m}_{B} {B}_{0} ( cos λ - 1 ) {\int }_{- \frac{π}{2 ω}}^{\frac{π}{2 ω}} sin ( 2 ω t ) dt = 0 \\ {\overline{T}}_{y} = - \frac{1}{2} {m}_{B} {B}_{0} ( cos λ - 1 ) {\int }_{- \frac{π}{2 ω}}^{\frac{π}{2 ω}} sin ( 2 ω t ) dt = 0\end{matrix} \qquad ( 4 )$$

From formula (4), the average moment received by the radially magnetized permanent magnet in the SURMF within a single period is the average moment T̅ₓ of rotating around ox₀ axis, and is called the coaxial following magnetic moment, and the average moment received in other directions is 0. Under the action of the coaxial following magnetic moment T̅ₓ, the radially magnetized permanent magnet deflects with the normal vector of the rotating magnetic field and finally reaches position 2, so that the central axis direction n_{B} of the radially magnetized permanent magnet is consistent with the direction of the normal vector n_{f} of the rotating magnetic field.

It can be seen that the coaxial following magnetic moment effect is expected to solve the problem of capsule posture drive and control. Therefore, according to the principle of the gyroscope dynamic balance, a dynamic control method of pure magnetic moment actuated capsule posture is proposed, which is different from the static balance control mode of capsule posture based on magnetic levitation. The high-speed rotating capsule robot driven by the rotating magnetic field has good dynamic balance characteristics and anti-interference capacity. The control process of capsule posture dynamic balance is simpler, rapid and convenient. By controlling the axis change of the rotating magnetic field, the capsule posture can be adjusted based on the coaxial following magnetic moment effect. In the study, it is also found that a single body structure capsule is difficult to achieve fixed point posture control, because the capsule is prone to roll during the posture adjustment. In fact, a capsule endoscope has the best inspection effect in the small intestine, but is likely more easily to roll away in 3D regions of the gastrointestinal (GI) tract, and difficult in posture control. The spherical capsule has the best rolling flexibility in the non-structural 3D regions of the gastrointestinal (GI) tract. In detection at the 3D regions of the gastrointestinal (GI) tract, to prevent the spherical robot from rolling during posture adjustment and ensure that the robot axis moves synchronously with the rotating magnetic field in situ, in combination with the flexibility and universality of the posture adjustment and turning of the spherical structure, in accordance with the posture adjustment stability and uniqueness of the coaxial following magnetic moment effect of the SURMF, a dual hemisphere capsule robot with active and passive modes is proposed in patent “a dual hemisphere capsule robot with active and passive modes and a posture adjustment and turning drive control method thereof” (patent authorization number: ZL201510262778.4) applied by Dalian University of Technology. The coupling of the SURMF and the radially magnetized magnet ring in the active hemisphere generates the coaxial following magnetic moment. The active hemisphere is rotated in term of idling around the passive hemisphere. The passive hemisphere is in an under-actuated state. The under-actuated hemisphere structure enhances the stability of the posture adjustment of the dual hemisphere capsule robot and the adaptability to non-structural environments, and the suspended posture adjustment at a fixed-point in the passive mode and the rolling locomotion in the active mode can be converted. The suspended posture adjustment of the dual hemisphere capsule at a fixed-point is realized: the active hemisphere is always on the top, and the passive hemisphere is on the bottom; the passive hemisphere is in a static state under the restriction of contact with the intestinal tract, the capsule will not roll, and the axis direction of the rotating magnetic field is changed (scanning and observation within a conical surface at an angle of about 30 degrees from the vertical direction of the gastrointestinal surface). Under the action of the coaxial following magnetic moment, the capsule axis and the rotating magnetic field axis move synchronously, and the “suspended” posture of the capsule can be adjusted arbitrarily to achieve panoramic observation. The capsule robot rolls along the bending direction of the intestinal tract: the azimuth angle of a space universal rotating magnetic vector is adjusted by a front image transmission device of the capsule robot to make the robot axis basically consistent with the direction of the bending normal line of the intestinal tract; and a rotating magnetic vector perpendicular to the bending direction of the intestinal tract is applied in the horizontal plane. Under the action of the coaxial following magnetic moment effect, the robot axis follows the direction of the horizontal rotating magnetic vector, and both the active hemisphere and the passive hemisphere come into contact with the lower wall of the intestinal tract. The coupled magnetic moment drives the active hemisphere to contact the lower wall of the intestinal tract to roll actively, and the under-actuated hemisphere rolls passively with the lower wall of the intestinal tract, so that the dual hemisphere capsule robot rolls by a limited distance along a bending direction in the intestinal tract.

To align and detect the bending direction of the intestinal tract by using a radio frequency transmitting image of the dual hemisphere capsule to realize the magnetic navigation of the capsule in the curved intestinal tract, in the invention patent “a dual image visual navigation method of a dual hemisphere capsule robot in a curved intestinal tract” (authorization number: ZL201910227499.2) applied by Dalian University of Technology, a specific navigation method using the radio frequency transmitting image is provided. In the method, firstly, the coaxial following characteristics of the axis of the dual hemisphere capsule robot and the rotation axis of the universal magnetic field are used, that is, the axis of the dual hemisphere capsule robot, the optical axis of a camera and the rotation axis of the universal magnetic field are all overlapped. The posture information of the dual hemisphere capsule robot is determined by combining two radio frequency transmitting images taken by a monocular camera at the same pitch angle and two different swing angles. Then, in combination with the posture information obtained, the visual navigation azimuth of the dual hemisphere capsule robot is deduced under the condition of a universal uniform rotating magnetic field. That is, by calculating the azimuth of the centroid of the dark area of the curved intestine image in a fixed coordinate system, the posture of the dual hemisphere capsule robot is adjusted to accurately align the bending direction of the intestinal tract, and the axis direction of the turning and rolling magnetic field of the dual hemisphere capsule robot is determined to realize the visually-assisted navigation operation of the dual hemisphere capsule robot in the intestinal tract.

To more conveniently control the posture of the capsule according to the coaxial following magnetic moment effect and realize the unity of the capsule axis and the magnetic field control rotation axis, it is necessary to realize the digital control of the azimuth of the SURMF by taking capsule swing angles and pitch angle as variables. In the invention patent “human-computer interactive control method for space universal rotating magnetic field” (patent authorization number: ZL 201610009285.4) applied by Dalian University of Technology, to overlap the SURMF with swing angle θ and pitch angle δ as rotation axes, the SURMF superposition formula of current form with two angles of swing angle θ and pitch angle δ as input variables in a latitude and longitude coordinate system is proposed:

$$[ \begin{matrix}{I}_{x} \\ {I}_{y} \\ {I}_{z}\end{matrix} ] = [ \begin{matrix}{I}_{0} \sqrt{1 - {sin}^{2} θ {cos}^{2} δ} sin ( ω t - {φ}_{x} ) \\ - {I}_{0} \sqrt{1 - {cos}^{2} θ {cos}^{2} δ} sin ( ω t + {φ}_{y} ) \\ {I}_{0} cos δ sin ( ω t + π / 2 )\end{matrix} ] \qquad ( 5 )$$

where o−xyz is the fixed coordinate system of the universal magnetic field; φₓ=arctan(tan θ sin δ), φ_{y}=arctan(cot θ sin δ); I₀ is the amplitude of sine current in tri-axis orthogonal square Helmholtz coils; ω is the angular frequency at which the sine signal current is applied; and the frequency at which the sine signal current is applied is f=2π/ω. The three-dimensional superposition problem of the SURMF is transformed into the two-dimensional superposition problem in a plane, and the swing angle and the pitch angle are separately controlled through two joysticks to realize the interactive control of low-dimensional separable variables.

Although the dual hemisphere capsule robot solves the conversion problem of the dual modes of suspended posture adjustment at a fixed-point and rolling locomotion. In the experiment, we have found that the active and passive hemisphere capsule robot still has the problems of small range of posture adjustment, poor adaptability to non-structural complex environments, etc., and especially has the contradiction between the accurate control of slow shift and poor stability, which affects the clinical application effect of gastrointestinal disease detection.

The posture adjustment at a fixed-point in the passive mode has the following problems: 1) The range of posture adjustment is limited, and all-round observation for the gastrointestinal wall of the lower part of the gastrointestinal tract cannot be realized. Generally, in observation operation, the posture adjustment angle of the dual hemisphere capsule is within the range of the conical surface at an angle of about 30 degrees from the vertical direction. If the posture adjustment angle is too large, the active hemisphere is easy to contact the gastrointestinal tissue and roll away, resulting in the failure of the fixed point posture adjustment. 2) The posture adjustment function of the capsule is limited in non-structural complex environments. When the capsule falls into the narrow concave region of the gastrointestinal tract, the active hemisphere is easier to contact the gastrointestinal tract, and the range of posture angle adjustment is smaller. Especially when the capsule is completely wrapped tightly in the gastrointestinal tissue, the posture adjustment function fails. In fact, in order to expand the functions of capsule sampling and fixed point drug spraying, the capsule must have the capacity of universal posture adjustment in the non-structural complex environments of the gastrointestinal tract, which is a very challenging research problem. 3) Eccentric vibration may affect the fixed point posture control accuracy and visual observation effect. The active hemisphere of the embedded radially magnetized magnet ring always rotates with the magnetic field during posture adjustment, and the active hemisphere increases the moment of inertia of the rotor. If there is eccentricity error of installation between the radially magnetized magnet ring and the active hemisphere, vibration will inevitably occur and affect the stability and accuracy of the capsule posture control.

The rolling locomotion in the active mode has the following problems: 1) There is a contradiction between posture stability and the slow and accurate displacement requirement. Because the rolling speed of the capsule is the same as the rotation speed of the magnetic field, too high rotation speed of the magnetic field is not conducive to the accurate control of the capsule displacement and observation. If the rotation speed of the magnetic field is low, the capsule robot has poor stability and difficulty in controlling the rolling direction. 2) The fault tolerant rolling function is limited in complex environments. When rolling in the curved intestinal tract, if the rolling direction is different from the bending direction of the intestinal tract, the dual hemisphere capsule will contact the intestinal wall and produce power parasitic phenomena such as slipping rolling or skidding, which will affect the capsule stability and the rolling locomotion direction and then affect the turning function. The turning adaptability needs to be improved. Although the visual system of the dual hemisphere capsule is fixedly connected with the passive hemisphere, the visual system and the passive hemisphere do not rotate during posture adjustment in the passive mode. However, when the capsule rolls in the active mode, the visual system and the passive hemisphere will roll together with the active hemisphere and directly affect the visual observation effect.

In view of the problems of small posture adjustment range and limited adaptability to complex environments of the dual hemisphere capsule, an all-round observation overall under-actuated spherical capsule robot controlled by the SURMF is proposed to apply for patent. Different from the dual hemisphere capsule structure with active hemisphere and under-actuated hemisphere, the overall capsule sphere completely adopts the under-actuated structure, that is, the radially magnetized NdFeB magnet ring is loaded into the inner cavity of the capsule sphere in a non-connected way, and the radially magnetized NdFeB magnet ring independently idles with the rotating magnetic field around the capsule axis and is completely suspended in the inner cavity filled with silicone oil in the capsule sphere. Under the drive of the coaxial following magnetic moment, although the under-actuated capsule sphere cannot rotate around the capsule axis (the capsule sphere is stationary due to the constraints of the concave region of the intestinal tract and the mucus), the capsule axis can roll synchronously with the rotation axis of the magnetic field. Therefore, the rolling over motion track planning of the capsule axis is controlled synchronously by the universal magnetic field to realize the all-round fixed point posture adjustment and rolling locomotion of the under-actuated capsule: when the capsule axis track is controlled to move within the conical surface (0˜±90°) at a certain angle with the axis direction perpendicular to the gastrointestinal wall surface, universal scanning and observation for the capsule posture can be realized; and when the capsule axis track is controlled to continuously roll over circularly in a vertical plane parallel to the bending direction of the intestinal tract, rolling scanning and observation for the capsule can be realized.

The proposed under-actuated spherical capsule significantly improves the all-round observation performance and the adaptability to non-structural environments: 1) The posture adjustment range of the capsule is significantly increased. Because the under-actuated capsule sphere does not roll when coming into contact with the gastrointestinal tissue, the scanning range is not limited and the capsule axis can roll over universally following the rotation axis of the magnetic field, or even realizes the all-round observation of the gastrointestinal wall at the lower part of the capsule. 2) The adaptability to complex intestinal environments is enhanced. The test shows that even if the capsule is wrapped in the gastrointestinal tissue or falls into the narrow concave region of the gastrointestinal tract, the coaxial following magnetic moment can completely overcome the resistance of the gastrointestinal wall and make the capsule axis roll over universally synchronously with the axis of the rotating magnetic field. Because the capsule adopts dynamic posture control, the gastrointestinal wall damping is more conducive to the stable control of the capsule control to realize the online all-round observation of the gastrointestinal tract. The whole sphere does not roll, and the visual observation effect is good, which lays a foundation for expanding the functions of sampling and fixed point drug spraying of the capsule. 3) The capsule has good vibration suppression effect, high accuracy of fixed point posture adjustment and locomotion, and good visual observation effect. The reason is that the NdFeB magnet ring that idles independently following the rotating magnetic field is completely suspended in the inner cavity filled with silicone oil in the capsule sphere, and visual modules and other electronic modules are completely embedded in the sphere. Because the under-actuated sphere does not rotate with the magnetic field, the visual observation effect is not affected, and especially the manufacturing and installation eccentricity of the devices in the sphere will not cause vibration. Therefore, the capsule stability and posture control precision are improved, and the visual observation effect is good. 4) The fault-tolerant rolling locomotion ability of the capsule in the non-structural complex curved intestinal environment is ensured. When rolling in the curved gastrointestinal tract, if the rolling direction is different from the bending direction of the gastrointestinal tract, the under-actuated sphere contacts with the intestinal wall and rolls adaptively, thereby avoiding the power parasitic phenomena such as slipping rolling or skidding, and improving the adaptability of the capsule to turning. 5) The contradiction between posture stability and slow and accurate displacement requirement is solved. The rolling speed of the capsule is irrelevant to the rotation speed of the rotating magnetic field, and the fast rotation speed of the magnetic field is conducive to capsule stability, robot observation and control. The rolling speed of the capsule is relevant to the rotation speed of the magnetic field axis, and the capsule posture stability and slow accurate shift can be independently controlled.

In fact, it is necessary to ensure that the under-actuated spherical capsule does not rotate and roll, and realize posture stability control, so as to realize universal fixed point scanning and rolling locomotion through rolling motion track control of the capsule axis. Because the NdFeB magnet ring that idles with the magnetic field is suspended in the ring sealing cavity filled with silicone oil in the capsule sphere, the fluid friction moment must be reduced in order to prevent the oil membrane from producing fluid friction moment and driving the capsule sphere to revolve (rotate) to avoid destroying the stability of the fixed position posture adjustment.

The “wedge effect” is used for the cylindrical capsule profile design in the invention patent “a capsule robot and a multi-wedge effect drive control method thereof” (patent authorization number: ZL200910306805.8) applied by Dalian University of Technology. Multiple wedge gaps are formed between the rotating capsule and the pipe wall in the axis section. Multiple wedge effects can increase the dynamic pressure of hydraulic oil membranes and reduce the viscous torque of fluid between the capsule and the pipe wall, thereby reducing the torsional effect on the pipe wall. In view of this, the under-actuated spherical capsule intended to apply for patent also increases the dynamic pressure of the hydraulic oil membranes formed by the contact surface of the NdFeB magnet ring and the inner cavity of the sphere by changing the profile of the inner cavity filled with silicone oil inside the capsule sphere, to avoid direct contact with the sphere shell, reduce the effect of viscous torque of fluid on the spherical capsule, and effectively prevent the magnetic ring from driving the capsule to revolve (rotate) and causing unstability.

As shown by the mastered data and the novelty search for project approval, at present, there is no relevant data reported in China and abroad that the under-actuated capsule can still realize all-round scanning and observation under the gastrointestinal tissue wrapping by using the coaxial following magnetic moment effect of the SURMF, so the present invention has prominent originality.

### SUMMARY

To improve the control performance of an active capsule in non-structurally complex environments, the present invention provides a magnetic field control method for all-round fixed position posture adjustment or rolling locomotion of the capsule through synchronous rolling over motion track planning of the capsule axis along with the rotation axis of the magnetic field under the direct guidance and drive of the coaxial following magnetic moment of a space universal rotating magnetic field (SURMF) by an overall spherical under-actuated capsule structure formed by loading a radially magnetized NdFeB magnet ring into a ring sealing cavity inside a capsule sphere in a full suspension mode.

The technical solution of the present invention is as follows:

An all-round observation under-actuated capsule robot comprises an under-actuated sphere 1 and a radially magnetized NdFeB magnet ring 2. Under the action of the SURMF, two functions of universal fixed point posture adjustment scanning observation and rolling locomotion of the under-actuated sphere 1 in the gastrointestinal tract are realized.

The under-actuated sphere 1 comprises an overall shell I, a camera module II, a radio frequency transmitting module III and a power supply battery 11. The power supply battery 11, the camera module II and the radio frequency transmitting module III are integrated into the under-actuated sphere 1 to realize the functions of power supply, image shooting, illumination and image transmission of the capsule. Because the under-actuated sphere 1 cannot rotate with the SURMF around the central axis of the capsule robot, the stability of dynamic posture adjustment and good visual observation effect of the capsule robot are ensured.

The overall shell I comprises a transparent end cover 3, an upper shell 4, a seal ring 5 and a lower shell 6. The transparent end cover 3, the upper shell 4 and the lower shell 6 are mutually embedded to ensure that the overall shape of the capsule robot is spherical and a ring sealing cavity is formed in the under-actuated sphere 1.

The composition of the ring sealing cavity is as follows: a cylinder is arranged in the center of the upper shell 4; the tops of the upper shell and the cylinder are fixed into a whole through connecting end surfaces; a gap between the cylinder and the inner wall of the upper shell 4 forms a ring groove; the inner wall of the upper shell 4 is used as an outer loop surface, and the outer wall of the cylinder is used as an inner loop surface; the outer loop surface is provided with a plurality of cylindrical bulges; the inner loop surface is smooth; the surface of the connecting end surface located on one side of the ring groove is provided with a plurality of spherical bulges; the seal ring 5 is embedded into the ring groove in the upper shell 4; and the end surface of the seal ring 5 opposite to the connecting end surface is provided with a plurality of spherical bulges to jointly form two end surfaces, the inner loop surface and the outer loop surface of the ring sealing cavity; the ring sealing cavity is filled with silicone oil; and the function of the seal ring 5 is to provide an inner end surface with a plurality of spherical bulges for the ring sealing cavity and prevent the leakage of the silicone oil.

The radially magnetized NdFeB magnet ring 2 is sleeved on the cylinder in the center of the upper shell 4, and is loaded into the ring sealing cavity filled with silicone oil in the under-actuated sphere 1 in a non-connected way; the under-actuated sphere 1 is completely in an under-actuated posture; and the under-actuated sphere 1 has good adaptability to the unstructured environment of the gastrointestinal tract, and is also the key to realize the function of all-round fixed point posture adjustment. When the capsule robot works in the SURMF, the radially magnetized NdFeB magnet ring 2 independently idles with the rotating magnetic field around the central axis of the capsule robot and is completely suspended in the ring sealing cavity filled with silicone oil in the under-actuated sphere 1; and the under-actuated sphere 1 of the capsule is in a static state.

Two end surfaces of the radially magnetized NdFeB magnet ring 2 and the two end surfaces with a plurality of bulges on the ring sealing cavity of the under-actuated sphere 1 respectively form two multi-wedge gaps; A multi-wedge gap is formed between the outer loop surface of the radially magnetized NdFeB magnet ring 2 and the outer loop surface with a plurality of bulges on the under-actuated sphere 1. Fluid multiple wedge effects are used to increase the dynamic pressure of a fluid oil membrane between the radially magnetized NdFeB magnet ring 2 under rotation and the ring sealing cavity and reduce the viscous torque of fluid of the under-actuated sphere 1. The radially magnetized NdFeB magnet ring 2 and the ring sealing cavity are coaxial, to avoid direct contact with the overall shell I and effectively prevent the radially magnetized NdFeB magnet ring 2 from driving the under-actuated sphere 1 to rotate and causing unstability of the capsule in an operation process.

The camera module II is composed of a camera element 7 and an LED illumination module 8 to realize the functions of photographing and illumination of the capsule robot. The cylinder in the center of the upper shell 4 is provided with a groove; the camera element 7 is integrally embedded into the groove; the LED illumination module 8 is installed above the camera element 7 and positioned by the cylinder surface; the transparent end cover 3 is located above the camera element 7; and the camera element 7 is used for photographing an external environment.

The radio frequency transmitting module III is composed of a radio frequency transmitter 9 and a radio frequency transmitting antenna 10 to realize the image transmission function of the capsule robot. The radio frequency transmitter 9 is embedded into the groove of the lower shell 6, and the bottom thereof is in contact with the power supply battery 11; and the radio frequency transmitting antenna 10 is embedded into a ring groove around the lower shell 6.

The power supply battery 11 supplies power for the camera element 7, the LED illumination module 8 and the radio frequency transmitter 9. The power supply battery 11 is embedded into the groove of the lower shell 6.

The magnetic control operation process for realizing two functions of universal fixed point scanning observation and rolling locomotion by the all-round observation under-actuated capsule robot is: under the action of the SURMF, under the condition that the radially magnetized NdFeB magnet ring 2 is driven by the coaxial following magnetic moment, although the under-actuated sphere 1 cannot rotate around the central axis of the capsule robot, the central axis of the under-actuated sphere 1 can roll synchronously with the rotation axis of the magnetic field. Therefore, the radially magnetized NdFeB magnet ring 2 can drive the axis of the under-actuated sphere 1 for synchronous rolling over motion track planning along with the rotation axis of the magnetic field to realize all-round fixed position posture adjustment or rolling locomotion of the capsule. The universal magnetic field is used for controlling the capsule robot axis to scan in sequence within a conical surface at a certain angle from a vertical direction or to make continuous circular rolling over in a vertical plane parallel to the bending direction of an environment to be measured, so as to respectively realize universal observation diagnosis of the capsule robot and bending locomotion along the bending direction under the internal constraints of the environment to be measured (such as the gastrointestinal tract).

The all-round scanning observation control process of the all-round observation under-actuated capsule robot is:
step 1: realizing initial calibration of the azimuth of the all-round under-actuated capsule robot: exerting the normal vector of the rotating magnetic field to be consistent with a ground vertical vector n₀; and based on the coaxial following magnetic moment effect, finally maintaining the capsule robot axis n_{B} the consistent with the normal vector of the rotating magnetic field;
step 2: realizing top observation diagnosis of the environment to be measured: when controlling the track of the capsule robot axis n_{B} to move and observe in sequence within a conical surface at a certain angle α with the ground vertical vector n₀, based on the coaxial following magnetic moment effect, controlling the capsule robot axis n_{B} to scan and observe in sequence along with the normal vector direction of the magnetic field; wherein the range of α is 0˜90°;
step 3: realizing bottom observation diagnosis of the environment to be measured: when controlling the track of the capsule robot axis n_{B} to move and observe in sequence within a conical surface at a certain angle −α with the ground vertical vector n₀, controlling the capsule robot axis n_{B} to scan and observe in sequence along with the normal vector direction of the magnetic field;
wherein the control process of linear and bending rolling locomotion of the all-round observation under-actuated capsule robot is:
step 1: completing visual detection of the bending direction of the environment to be measured: controlling the track of the capsule robot axis n_{B} to scan and observe in sequence within a conical surface at a certain angle ±α with the ground vertical vector n₀, until the bending direction of the environment to be measured is basically aligned; and determining the bending direction of the environment to be measured through a visual positioning method; wherein the range of α is 0˜90°;
step 2: realizing rolling locomotion in a linear environment to be measured: the bending direction of the environment to be measured, determined in step 1, being the rolling locomotion direction of the capsule robot; when a rolling vector of the linear environment to be measured is nₛₐ, continuously changing the azimuth of the normal vector n_{f} of the rotating magnetic field in a vertical plane V₁ formed by a ground vertical vector n₀ and a linear rolling vector nₛₐ, i.e., controlling the capsule robot axis n_{B} to make continuous circular rolling in the vertical plane V₁ to realize the function of rolling locomotion of the capsule robot along a straight line under the internal constraint of the environment to be measured;
step 3: realizing rolling locomotion in a bending environment to be measured: the bending direction of the environment to be measured, determined in step 1, being the rolling locomotion direction of the capsule robot; when a bending vector of the bending environment to be measured is n_{sb}, continuously changing the azimuth of the normal vector n_{f} of the rotating magnetic field in a vertical plane V formed by a ground vertical vector n₀ and a bending rolling vector n_{sb}, to control the capsule robot axis n_{B} to make continuous circular rolling in the vertical plane V.

The present invention has the following effects and benefits:

In the all-round observation under-actuated spherical capsule robot controlled by the SURMF, the radially magnetized NdFeB magnet ring is loaded into the ring sealing cavity filled with silicone oil in the capsule sphere in a completely suspended mode; the overall capsule sphere uses the under-actuated structure; the radially magnetized NdFeB magnet ring idles individually with the rotating magnetic field and is completely suspended in the center of the ring sealing cavity filled with silicone oil in the capsule sphere. Fluid multi-wedge effects are used to increase the dynamic pressure of a fluid oil membrane between the radially magnetized NdFeB magnet ring and the ring sealing cavity and reduce the viscous torque of fluid of the under-actuated sphere by changing the end surface around the ring sealing cavity filled with silicone oil and the profile of the loop surface. The radially magnetized NdFeB magnet ring idles individually and is completely suspended in the center of the ring sealing cavity of the capsule. The radially magnetized NdFeB magnet ring and the ring sealing cavity are coaxial, to effectively avoid driving the capsule sphere to rotate and destabilize. Under the direct guidance and drive of the coaxial following magnetic moment of the SURMF, all-round fixed position posture adjustment or rolling locomotion is realized through the control of synchronous rolling over motion of the capsule axis along with the rotation axis of the magnetic field.

The present invention increases the scanning range of the capsule in human complex environments, realizes the all-round observation of the gastrointestinal wall at the lower part of the capsule in various environments to be measured, enhances the adaptability to complex intestinal environments, still can realize the all-round observation if the capsule is wrapped in the gastrointestinal tissue or falls into the narrow concave region of the gastrointestinal tract, and has good fault tolerant and adaptive rolling locomotion capabilities in the curved intestinal tract, high accuracy of fixed point posture adjustment of the capsule, rolling locomotion stability, good visual observation effect and good application prospect.

### DESCRIPTION OF DRAWINGS

FIG. 1 is a schematic diagram of a “coaxial following magnetic moment effect” of the SURMF.

FIG. 2 is a schematic diagram of a magnetic field driving device and a control system of an under-actuated spherical capsule robot.

FIG. 3 is an overall structural schematic diagram of an under-actuated spherical capsule robot of the present invention.

FIG. 4 is an internal structural sectional view of an under-actuated spherical capsule robot of the present invention.

FIG. 5(a) is a schematic diagram of installation composition of a ring sealing cavity in an under-actuated capsule;

FIG. 5(b) is a sectional view of a ring sealing cavity in an under-actuated capsule.

FIG. 6 is a schematic diagram of all-round scanning magnetic control operation of an under-actuated capsule robot.

FIG. 7 is a schematic diagram of rolling locomotion and bending magnetic control operation of an under-actuated capsule robot.

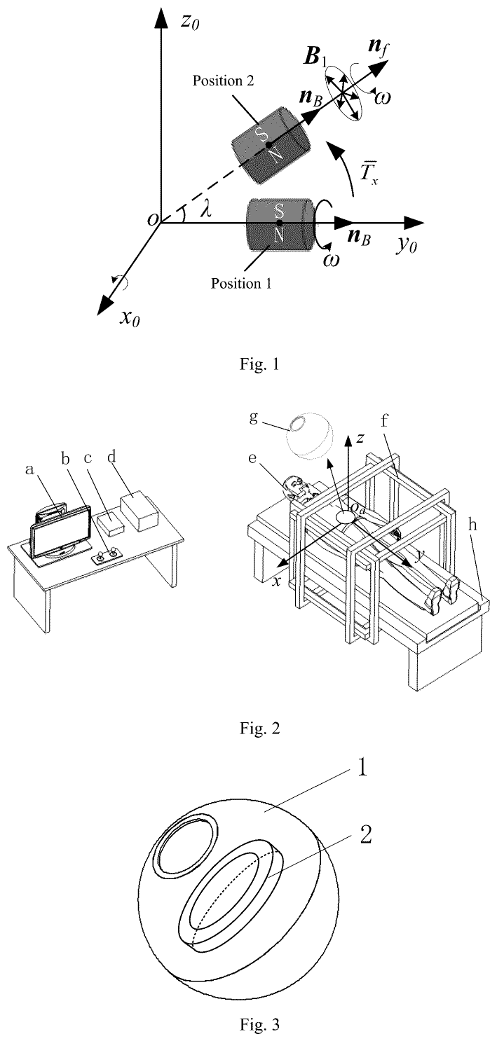

In the figures: o-x₀y₀z₀ is a ground fixed coordinate system; o−xyz is a universal magnetic field fixed coordinate system; position 1—initial posture of radially magnetized permanent magnet 2; position 2—target posture finally achieved by radially magnetized permanent magnet 2 after exerting the rotating magnetic field of normal vector azimuth n_{f};

a—man-machine display interface; b—control handle; c—wireless image receiver; d—magnetic field driver; e—examinee; f—TOSHC device for magnetic field superposition; g—all-round observation under-actuated spherical capsule robot; h—hospital bed;

I—overall shell; II—camera module; III—radio frequency transmitting module;

1—under-actuated sphere; 2—radially magnetized NdFeB magnet ring; 3—transparent end cover; 4—upper shell; 5—seal ring; 6—lower shell; 7—camera element; 8—LED illumination module; 9—radio frequency transmitter; 10—radio frequency transmitting antenna; 11—power supply battery.

### DETAILED DESCRIPTION

Detailed description of the present invention is described below in detail in combination with accompanying drawings and the technical solution.

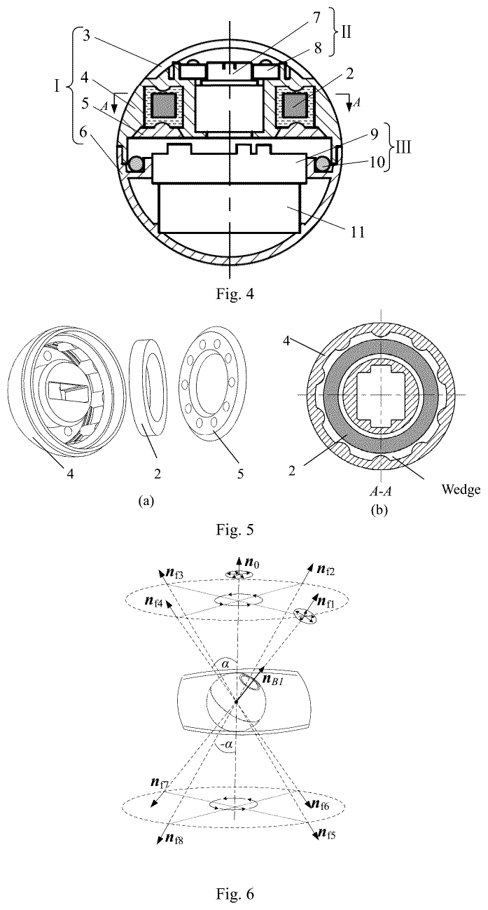

In combination with FIGS. 3-4, 5(a) and 5(b), the installation process of an under-actuated capsule robot is as follows:
Step 1: putting a radially magnetized NdFeB magnet ring 2 into a ring groove of an upper shell 4, and filling the ring groove with silicone oil lubricating oil; embedding a seal ring 5 into the ring groove of the upper shell 4, and bonding with seal glue to form a ring sealing cavity; and respectively forming three multi-wedge gaps around the radially magnetized NdFeB magnet ring 2 and the ring sealing cavity.
Step 2: embedding a camera element 7 into a groove of the upper shell 4; installing an LED illumination module 8 above the camera element 7; making a central hole thereof coaxial with bulges above the camera element 7; and making a power supply wire thereof penetrate from a rectangular hole in the center of the upper shell 4.
Step 3: embedding a power supply battery 11 into a circular groove of a lower shell 6; embedding a radio frequency transmitter 9 into a rectangular groove of the lower shell 6, and making the bottom thereof in contact with the power supply battery 11; and embedding the radio frequency transmitting antenna 10 into a ring gap around the lower shell 6.
Step 4: connecting a power line of the power supply battery 11 with a camera module II and a radio frequency transmitting module III, and connecting a signal line of the camera module II with a signal line of the radio frequency transmitting module III; and finally, mutually embedding a transparent end cover 3, the upper shell 4 and the lower shell 6 so that the overall capsule robot is sealed to form the shape of the overall capsule robot to complete the assembly of the overall capsule robot.

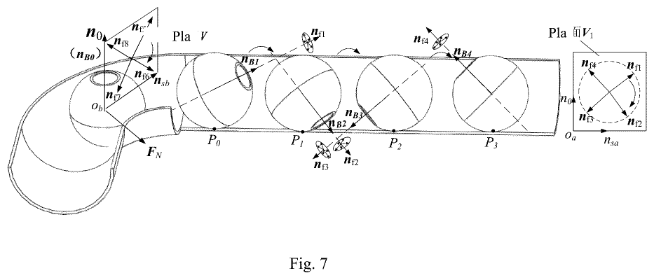

The detection process for human gastrointestinal diseases by the capsule robot is illustrated in combination with FIGS. 2-7 below by taking the gastrointestinal tract as an environment to be measured:
Step 1: swallowing an all-round observation under-actuated spherical capsule robot g by an examinee e, and lying on a hospital bed h; and adjusting the position of the hospital bed h to ensure that the examinee e is located in a central region of a TOSHC device f for magnetic field superposition.
Step 2: starting the TOSHC device f for magnetic field superposition through a magnetic field driver d; moving the under-actuated spherical capsule robot g in a human body; making a video recording in real time for the gastrointestinal tract in the human body through a camera module II; and transmitting image signals through a radio frequency transmitting module III.
Step 3: receiving the image signals through a wireless image receiver c, and displaying on a man-machine display interface a.
Step 4: observing the gastrointestinal tract in the body of the examinee e by an examiner through pictures, and adjusting the direction of the magnetic field through a control handle b to align with the bending direction of the gastrointestinal tract to control the under-actuated spherical capsule robot g to move to a designated observation point; adjusting the posture of the capsule at the designated observation point to realize all-round observation; and repeating the above process to realize ergodic inspection of the capsule robot in the gastrointestinal tract.

The all-round scanning observation control process of the under-actuated capsule robot is illustrated in combination with FIGS. 2, 3 and 6:
Step 1: realizing initial calibration of the azimuth of the under-actuated capsule robot: exerting the normal vector of the rotating magnetic field to be consistent with a ground vertical vector n₀ through the control handle b; and based on the coaxial following magnetic moment effect, finally maintaining the capsule robot axis n_{B} consistent with the normal vector of the rotating magnetic field.
Step 2: realizing top observation diagnosis of gastrointestinal tissue: when controlling the track of the capsule robot axis n_{B} to move and observe in sequence within a conical surface at a certain angle α (0˜90°) with the ground vertical vector n₀ through the control handle b, based on the coaxial following magnetic moment effect, controlling the capsule robot axis n_{B} to scan and observe in sequence along with the normal vector direction n_{f1}→n_{f2}→n_{f3}→n_{f4} of the magnetic field to realize the top observation diagnosis of the gastrointestinal tissue, as shown in FIG. 6.
Step 3: realizing bottom observation diagnosis of the gastrointestinal tissue: when controlling the track of the capsule robot axis n_{B} to move and observe in sequence within a conical surface at a certain angle −α (0˜−90°) with the ground vertical vector n₀ through the control handle b, controlling the capsule robot axis n_{B} to scan and observe in sequence along with the normal vector direction n_{f5}→n_{f6}→n_{f7}→n_{f8} of the magnetic field to realize the bottom observation diagnosis of the gastrointestinal tissue, as shown in FIG. 6.

The control processes of linear and bending rolling locomotion of the under-actuated capsule robot are illustrated respectively in combination with FIGS. 2, 3 and 7:
Step 1: completing visual detection of the bending direction of the gastrointestinal tract: controlling the track of the capsule robot axis n_{B} to scan and observe in sequence within a conical surface at a certain angle ±α (0˜±90°) with the ground vertical vector n₀ through the control handle b, until the bending direction of the gastrointestinal tract is basically aligned; and determining the bending direction of the gastrointestinal tract through a visual positioning method.
Step 2: realizing rolling locomotion in a linear gastrointestinal tract: the bending direction of the gastrointestinal tract, determined in step 1, being the rolling locomotion direction of the capsule robot; when a rolling vector of the linear gastrointestinal tract is nₛₐ, continuously changing the azimuth n_{f1}→n_{f2}→n_{f3}→n_{f4} of the normal vector n_{f} of the rotating magnetic field in a vertical plane V₁ (oₐ−n₀nₛₐ) formed by a ground vertical vector n₀ and a linear rolling vector nₛₐ, i.e., controlling the capsule robot axis n_{B} to make continuous circular rolling in the vertical plane V₁ to realize the function (P₀-P₁-P₂-P₃) of rolling locomotion of the capsule robot along a straight line oₐnₛₐ under the internal constraint environment of the gastrointestinal tract. In this process, the capsule can stop rolling at any time as required for all-round fixed point observation.
Step 3: realizing rolling locomotion in a curved gastrointestinal tract: the bending direction of the gastrointestinal tract, determined in step 1, being the rolling locomotion direction of the capsule robot; when a bending vector of the gastrointestinal tract is n_{sb}, continuously changing the azimuth n_{f5}→n_{f6}→n_{f7}→n_{f8} of the normal vector n_{f} of the rotating magnetic field in a vertical plane V (o_{b}−n₀n_{sb}) formed by a ground vertical vector n₀ and a bending rolling vector n_{sb}, to control the capsule robot axis n_{B} to make continuous circular rolling in the vertical plane V. Because an under-actuated structure has good adaptability to non-structural environments, when rolling locomotion in the curved gastrointestinal tract, if the rolling direction is different from the bending direction of the gastrointestinal tract, the under-actuated sphere 1 comes into contact with the intestinal wall and rolls adaptively, to avoid power parasitic phenomena such as slipping rolling or skidding. Under the boost of the intestinal force F_{N}, the capsule robot can turn, thereby improving the turning adaptability of the capsule. The capsule robot can be controlled to smoothly pass through the curved gastrointestinal tract.

1. An all-round observation under-actuated capsule robot, comprising an under-actuated sphere (1) and a radially magnetized NdFeB magnet ring (2), wherein
the under-actuated sphere (1) comprises an overall shell (I), a camera module (II), a radio frequency transmitting module (III) and a power supply battery (11); the power supply battery (11), the camera module (II) and the radio frequency transmitting module (III) are integrated into the under-actuated sphere (1) to realize the functions of power supply, image shooting, illumination and image transmission of the capsule;
the overall shell (I) comprises a transparent end cover (3), an upper shell (4), a seal ring (5) and a lower shell (6); the transparent end cover (3), the upper shell (4) and the lower shell (6) are mutually embedded to ensure that the overall shape of the capsule robot is spherical and a ring sealing cavity is formed in the under-actuated sphere (1);
the composition of the ring sealing cavity is as follows: a cylinder is arranged in the center of the upper shell (4); the tops of the upper shell and the cylinder are fixed into a whole through connecting end surfaces; a gap between the cylinder and the inner wall of the upper shell (4) forms a ring groove; the inner wall of the upper shell (4) is used as an outer loop surface, and the outer wall of the cylinder is used as an inner loop surface; the outer loop surface is provided with a plurality of cylindrical bulges; the inner loop surface is smooth; the surface of the connecting end surface located on one side of the ring groove is provided with a plurality of spherical bulges; the seal ring (5) is embedded into the ring groove in the upper shell (4); and the end surface of the seal ring (5) opposite to the connecting end surface is provided with a plurality of spherical bulges to jointly form two end surfaces, the inner loop surface and the outer loop surface of the ring sealing cavity; the ring sealing cavity is filled with silicone oil;
the radially magnetized NdFeB magnet ring (2) is sleeved on the cylinder in the center of the upper shell (4), and is loaded into the ring sealing cavity filled with silicone oil in the under-actuated sphere (1) in a non-connected way; the under-actuated sphere (1) is completely in an under-actuated posture; when the capsule robot works in a space universal rotating magnetic field (SURMF), the radially magnetized NdFeB magnet ring (2) independently idles with the rotating magnetic field around the central axis of the capsule robot and is completely suspended in the ring sealing cavity filled with silicone oil in the under-actuated sphere (1); and the under-actuated sphere (1) is in a static state;
two end surfaces of the radially magnetized NdFeB magnet ring (2) and the two end surfaces with a plurality of bulges on the ring sealing cavity of the under-actuated sphere (1) respectively form two multi-wedge gaps; a multi-wedge gap is formed between the outer loop surface of the radially magnetized NdFeB magnet ring (2) and the outer loop surface with a plurality of bulges on the under-actuated sphere (1);
the camera module (II) is composed of a camera element (7) and an LED illumination module (8) to realize the functions of photographing and illumination of the capsule robot; the cylinder in the center of the upper shell (4) is provided with a groove; the camera element (7) is integrally embedded into the groove; the LED illumination module (8) is installed above the camera element (7) and positioned by the cylinder surface; the transparent end cover (3) is located above the camera element (7); the camera element (7) is used for photographing an external environment;
the radio frequency transmitting module (III) is composed of a radio frequency transmitter (9) and a radio frequency transmitting antenna (10) to realize the image transmission function of the capsule robot; the radio frequency transmitter (9) is embedded into the groove of the lower shell (6), and the bottom thereof is in contact with the power supply battery (11); and the radio frequency transmitting antenna (10) is embedded into a ring groove around the lower shell (6);
the power supply battery (11) supplies power for the camera element (7), the LED illumination module (8) and the radio frequency transmitter (9); the power supply battery (11) is embedded into the groove of the lower shell (6).

2. An axis rolling-over locomotion method by magnetic field control for the all-round observation under-actuated capsule robot, using the all-round observation under-actuated capsule robot of claim 1 and used for realizing two functions of universal fixed point scanning observation and rolling locomotion, wherein a magnetic control operation process is: under the action of the SURMF, under the condition that the radially magnetized NdFeB magnet ring (2) is driven by the coaxial following magnetic moment, although the under-actuated sphere (1) cannot rotate around the central axis of the capsule robot, the central axis of the under-actuated sphere (1) can roll synchronously with the rotation axis of the magnetic field; therefore, the radially magnetized NdFeB magnet ring (2) drives the axis of the under-actuated sphere (1) for synchronous rolling over motion track planning along with the rotation axis of the magnetic field to realize all-round fixed position posture adjustment or rolling locomotion of the capsule; the universal magnetic field is used for controlling a capsule axis to scan in sequence within a conical surface at a certain angle from a vertical direction to realize universal observation diagnosis of the capsule robot for the interior of an environment to be measured; the universal magnetic field is used for controlling a capsule robot axis to make continuous circular rolling over in a vertical plane parallel to the bending direction of the environment to be measured, so that the capsule robot conducts bending locomotion along the bending direction under the internal constraints of the environment to be measured.

3. The axis rolling-over locomotion method by magnetic field control for the all-round observation under-actuated capsule robot according to claim 2, wherein the all-round scanning observation control process of the all-round observation under-actuated capsule robot is:
step 1: realizing initial calibration of the azimuth of the all-round under-actuated capsule robot: exerting the normal vector of the rotating magnetic field to be consistent with a ground vertical vector n₀; and based on the coaxial following magnetic moment effect, finally maintaining the capsule robot axis n_{B} consistent with the normal vector of the rotating magnetic field;
step 2: realizing top observation diagnosis of the environment to be measured: when controlling the track of the capsule robot axis n_{B} to move and observe in sequence within a conical surface at a certain angle α with the ground vertical vector n₀, based on the coaxial following magnetic moment effect, controlling the capsule robot axis n_{B} to scan and observe in sequence along with the normal vector direction of the magnetic field; wherein the range of a is 0˜90°;
step 3: realizing bottom observation diagnosis of the environment to be measured: when controlling the track of the capsule robot axis n_{B} to move and observe in sequence within a conical surface at a certain angle −α with the ground vertical vector n₀, controlling the capsule robot axis n_{B} to scan and observe in sequence along with the normal vector direction of the magnetic field.

4. The axis rolling-over locomotion method by magnetic field control for the all-round observation under-actuated capsule robot according to claim 2, wherein the control process of linear and bending rolling locomotion of the all-round observation under-actuated capsule robot is:
step 1: completing visual detection of the bending direction of the environment to be measured: controlling the track of the capsule robot axis n_{B} to scan and observe in sequence within a conical surface at a certain angle ±α with the ground vertical vector n₀, until the bending direction of the environment to be measured is basically aligned; and determining the bending direction of the environment to be measured through a visual positioning method; wherein the range of α is 0˜90°;
step 2: realizing rolling locomotion in a linear environment to be measured: the bending direction of the environment to be measured, determined in step 1, being the rolling locomotion direction of the capsule robot; when a rolling vector of the linear environment to be measured isnₛₐ, continuously changing the azimuth of the normal vector n_{f} of the rotating magnetic field in a vertical plane V₁ formed by a ground vertical vector n₀ and a linear rolling vector nₛₐ, i.e., controlling the capsule robot axis n_{B} to make continuous circular rolling in the vertical plane V₁ to realize the function of rolling locomotion of the capsule robot along a straight line under the internal constraint of the environment to be measured;
step 3: realizing rolling locomotion in a bending environment to be measured: the bending direction of the environment to be measured, determined in step 1, being the rolling locomotion direction of the capsule robot; when a bending vector of the bending environment to be measured is n_{sb}, continuously changing the azimuth of the normal vector n_{f} of the rotating magnetic field in a vertical plane V formed by a ground vertical vector n₀ and a bending rolling vector n_{sb}, to control the capsule robot axis n_{B} to make continuous circular rolling in the vertical plane V.
